(19) United States
(12) Patent Application Publication
(10) Pub. No.: US 2004/0071754 A1
(43) Pub. Date: Apr. 15, 2004

(54) Oral contact sanitizing wipes (ORCO Sanitizing wipes)

(76) Inventor: Lorraine Mary Jupiter, New Orleans, LA (US)

Correspondence Address: LORRAINE JUPITER, 7421 ARBOR DR., NEW ORLEANS, LA, 70126, US

(21) Appl. No.: 10/217,306

(22) Filed: Oct. 15, 2002

(51) Int. Cl. A61K 9/70

(52) U.S. Cl. 424/443

(57) ABSTRACT

Oral Contact Sanitizing wipes are wet disposable cloths used to clean in or around the mouth, and/or items that come into contact with the mouth (e.g., teething rings, toys, medicine dispensers, etc.). Unlike other baby wipes, the Oral Contact Sanitizing wipes are especially formulated for oral use. Also available in fruit flavors.

Oral contact sanitizing wipes are wet disposable cloths used to clean in or around the mouth, and/or items that come into contact with the mouth (e.g., teething rings, toys, medicine dispensers, etc.). Also available in fruit flavors. The chemicals used for this product is composed of a germ killing and sanitizing formula.

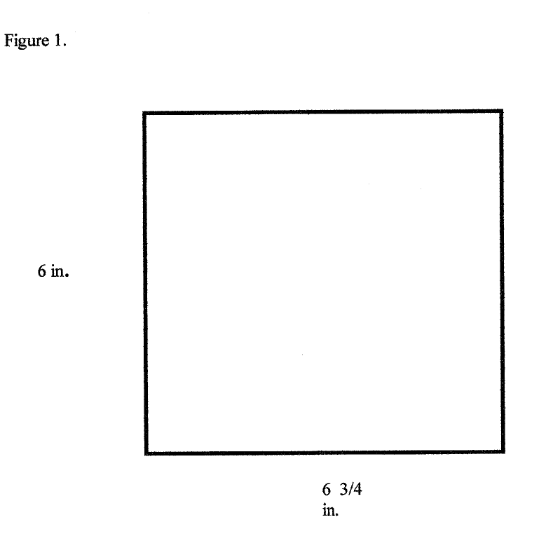

FIG. 1. shows an overall view of the invention. The cloth looks exactly the same from both the front view and the back view. The entire dampened cloth measures 6 in. 6¾ in (15.2 cm×17.1 cm).

1. I claim that this product is a specially formulated baby wipe used to clean in or around the mouth and/or objects that come into contact with the mouth (e.g., teething rings, toys, medicine dispensers, etc.).
